(19) United States
(12) Patent Application Publication
(10) Pub. No.: US 2022/0401293 A1
(43) Pub. Date: Dec. 22, 2022

(54) IMPROVED SEXUAL AID DEVICE AND METHOD

(71) Applicant: Lawrence David Mark, Christ Church (BB)

(72) Inventor: Lawrence David Mark, Christ Church (BB)

(21) Appl. No.: 17/772,361

(22) PCT Filed: Oct. 30, 2020
(86) PCT No.: PCT/IB2020/000944

§ 371 Date: Apr. 27, 2022

Related U.S. Application Data
(60) Provisional application No. 62/928,828, filed on Oct. 31, 2019.

(51) Int. Cl. A61H 19/00; A61F 5/41; A61H 23/02

(52) U.S. Cl. CPC A61H 19/50; A61H 19/44; A61F 5/41; A61H 19/34; A61H 23/02; A61F 2005/411; A61H 2201/0103; A61H 2201/163; A61H 2201/165; A61H 2201/0107

(57) ABSTRACT

An improved sexual aid device, the device including a semi-rigid base member for insertion into a woman's vagina. The semi-rigid flexible base member has a shape generally conforming to the shape of a male penis, and a top section secured to the semi-rigid base member. The top section includes an inflatable bladder, a foam member or a removable member. The top section receives the male penis. The device also may include a second curved external member which is a continuous extension from the base member, such that the device can simultaneously stimulate a woman's vagina and clitoris. Alternatively, the top section may include a removable member, and the device may include one more stimulation means.

### CROSS REFERENCE TO RELATED APPLICATIONS

This application claims the benefit of, and priority from, the Provisional Application Ser. No. 62/928,828, filed on Oct. 31, 2019.

### STATEMENT REGARDING FEDERALLY SPONSORED RESEARCH OR DEVELOPMENT

N/A

### BACKGROUND OF THE INVENTION

### 1. Field of the Invention

The present invention relates generally to devices for increasing human sexual arousal and pleasure, and more particularly to improved methods and devices for occupying the interior space of a human vaginal cavity for enhanced penile and vaginal stimulation during sexual intercourse.

### 2. Description of Related Art

In the relevant industry relating to sexual aid devices there are numerous and varied references illustrating and describing products to improve and assist with sexual intercourse, as well as sex toys, to enhance the experience, reach climax and achieve orgasm. These product designs range from relatively simple to the complex, and include diverse features such as multiple cams for expansion and contraction, programmable electronics, audio and visual features, attachments, fingers, vibration means, flexible and pliable materials and the like.

For example, U.S. Pat. No. 8,622,891 illustrates sexual aid devices to provide increased sexual sensation in either heterosexual or homosexual intercourse characterized by a structure maintaining the male penis in pressure contact with the sensitive members of the female vagina or male anus. The devices include a pliable material and penis support with a U-shaped channel, and the related U.S. Pat. No. 9,044,331 includes a cavity sized to hold a vibrator within the device to provide additional sexual stimulation.

U.S. Pat. No. 4,429,689 discloses a sex aid device for males having a tubular member that includes upper and lower longitudinally extending relatively inelastic but flexible supports and connecting elastic flexible sides. The tubular member is adapted to extend from the base of a penis to the glans penis, and the bottom support extending forward and forming a relatively inelastic spoon-like member for receiving the bottom of the glans penis to be exposed to tactile stimulation. The spoon-like member has a forward-extending smooth tip of soft compressible elastic material formed thereon, and the soft material is described as including foam. The device further preferably has upper and lower transverse ridges of smooth-surfaced compressible elastic material at the upper and lower lengths of the tubular member and one or two transverse side slits intermediate the length of the tubular member to aid in insertion of a non-erect penis. This device includes an attachment means for the body to support the unit.

U.S. Pat. No. 9,237,983 relates to a dual-penetration anal sex toy for couples. For heterosexual couples, this toy is designed to enhance vaginal penetration with an anal-to-anal connection. A cock ring or means for attachment may fix the device against the wearer's perineum and act as a pivot-point to transfer the thrusting motion of the wearer directly to a prostate massaging butt plug. Because of the pivot point and possible simultaneous anal penetration both partners can feel a direct connection with the anal movements of the other. This device also includes a plug and air valve for inflation.

U.S. Patent Application Publication No. 2012/0316388 discloses an adult toy which includes an elongated elastic body which has an inner space defined, and an opening defined at one end. A sealing device or a one-way device is disposed at the opening to seal the inner space. The user can make fluid flow through the sealing device or the one-way device into the inner space to inflate the elastic body, or release the fluid out of the inner space to deflate the elastic body. When the fluid is completely released, the adult toy is greatly reduced in size and is easy to store or carry. The elastic body may connect with a reservoir. The fluid received in reservoir can be forced to enter the elastic body and the fluid received in the elastic body can be released to the reservoir, thereby adjusting the stiffness and the size of the elastic body. The fluid can be air, water or oil.

U.S. Patent Application Publication No. 2013/0253264 describes a device for promoting and increasing sexual arousal, comprising a vibration unit. The device includes a rear ring and a front ring, which annularly surround a penis in the front and rear regions of the penis. The two rings are connected to each other by means of an intermediate part, which partially covers the penis between the rings. A vibration unit is integrated within the device. The device is made of an elastic, skin-compatible, slippery material. The device acts as a holster, includes vibrators, different materials and stiffening means.

U.S. Patent Application Publication No. 2016/0000642 relates to a relatively complex device including photo stimulation, various vibrators, sensory features, and components.

However, the above references are all distinct in terms of structure and function, and do not address the instant invention, in terms of Applicant's designs for the Improved Sexual Aid Device and Method. Applicant's prior U.S. Pat. No. 8,512,226 relates to a sexual aid and method for using the aid in sexual intercourse, and illustrates an aid that features a member that is removably insertable into a vagina of a human female. The insertable member occupies space within the vaginal cavity and thereby reduces the volume of unoccupied space inside the vagina. By leaving the insertable member in the vagina during sexual intercourse the female can experience a more pleasurable, fuller sensation within the vagina. Likewise, the male can experience a more pleasurable sensation of increased tightness and pressure against the penis while the penis is inserted within the vagina. This insertable member can be a unitary, generally solid, elongated member, and includes a tapered end and a bulbous end. During intercourse, when the male inserts his penis into the vagina, the decreased volume or free space available between the insertable member and an anterior portion of the vaginal wall provides the male with an increased sensation of tightness within the vagina, and thus, greater stimulation and pleasure. The thickened bulbous shape of the bulbous end of the insertable member as well as the diverging tip of the bulbous end stimulate the nerves contained within the vaginal wall to provide increased stimulation of the female during intercourse. The female also experiences a sensation of greater fullness during intercourse when both the male's penis and the insertable member are inserted within the vagina. By reducing the void space in the vaginal cavity in women, and particularly in women having a vaginal void with a volume greater than the average female, the insertion member can increase the tactile pressure on the male's penis and against the vaginal wall, thereby, enhancing stimulation of the couple in coitus.

Applicant's instant invention, and several improvements to Applicant's prior patent and designs, relate to new components and operation, including eliminating prior bulbous tips and penis troughs, and designing a product with the addition of a memory foam, air or detachable member, an air valve means to release air from either the foam version or an air inflatable version, one or more vibration, rotation, and pulsation means housed in the devices, and related components and features. It provides substantial improvements in stimulating and fulfilling the sexual experience of the partners, and further allows for an improved, accommodating and conforming fit and comfort for a man's penis utilizing the device, while simultaneously providing the benefits of reducing any vaginal void during intercourse.

Accordingly, there is a need in the art for a better designed, structurally and functionally sound, improved and more efficient sexual aid device and method of using the device.

It is, therefore, to the effective resolution of the aforementioned problems and shortcomings of the prior art that the present invention is directed. However, in view of the sexual aids and toys in existence at the time of the present invention, it was not obvious to those persons of ordinary skill in the pertinent art as to how the identified needs could be fulfilled in an advantageous manner.

### SUMMARY OF THE INVENTION

The present invention provides various embodiments of any improved sexual aid product and methods of using the product during sexual intercourse. The device includes the method of inserting and occupying the space within a woman's vagina, or first attaching the device under a man's penis, and using elastic ring at the base of the device or alternatively a condom over the product and the penis. The improved sex aid provides a greater sensation of increased fullness to the woman, and simultaneously a greater sensation of tightness and friction for the man during sexual intercourse. The improved product thereby enhances stimulation, sexual arousal and satisfaction for both the man and woman.

A first embodiment includes a rigid elongated base, generally spoon shaped and to conform to a man's penis, and an upper component attached to the base, the top section being made of memory foam or an air bladder. There is a valve for inflating the foam or bladder, and the base may have a silicone coating or other desired application. This device may function as a solo sex stimulant or toy, or for use with a couple during sex. The device can be inserted into the woman before the man enters, and thereafter the man will slide into the vagina on top of the foam. Alternatively, the man can first place his penis onto the memory foam or air bladder before entering the vagina, and thereafter enter with both in place. The device used in conjunction with the male penis creates a tighter fit for the man, a greater feel for the woman, and enhanced sensation and pleasure for both.

A second embodiment comprises a member having first section for insertion into the woman's vagina, and a second continuous and curved external lower section or finger member which remains outside the vagina for stimulating the clitoris. The finger member may have an enlarged end and during sex, the finger member is designed to be positioned in front of the male testicles and contacting the clitoris, however not in the missionary position because it would be above the male's penis, but would do so in the male behind position. Also included is an air valve for inflating a bladder or the use of memory foam secured atop the base member. These devices also include one or more vibrators selectively positioned about the aid, and control means for activation, and a removable or rechargeable battery power source.

A third embodiment relates to a member similar in design to the second embodiment, further adding a removable generally rigid top section above the base, and adapter for charging the battery. This sex aid can be used by a person solo, or by removing the top section to accommodate a man, and using the aid with a man having the penis on top of the device during sex as described above.

A forth embodiment comprises a design similar to the second embodiment, however the vibrator on the curved finger member for stimulating the clitoris area is replaced with a clitoral air flow unit for providing stimulating air flow and associated benefits.

A fifth embodiment is similar to the forth embodiment, but further provides a removable generally rigid top section above the base in place of the memory foam or air bladder.

A sixth embodiment is similar to the forth embodiment, however the clitoris air flow unit is replaced with clitoral pedal unit for stimulation.

A seventh embodiment is similar to the sixth embodiment, but further provides a removable generally rigid top section above the base in place of the memory foam or air bladder.

Accordingly, it is an object of the present inventions to provide improved sexual aid devices and methods of use.

It is another object of the present inventions to provide improved sexual aid devices and methods of use with structurally sound, operationally superior and cost effective mechanical and electronic designs and components.

It is another object of the present inventions to provide improved sexual aid devices and methods of use that are adaptable and feature alternative configurations for the devices as desired, and devices substantially enhancing and fulfilling sexual pleasure.

It is yet another object of the present inventions to provide improved sexual aid devices that are simple to use, safe for humans, and easy to maintain.

Finally, it is an object of the present inventions to provide improved sexual aid devices and methods of use having all of the above features and objects.

In accordance with these and other objects which will become apparent hereinafter, the instant invention will now be described with particular reference to the accompanying drawings.

### BRIEF DESCRIPTION OF THE DRAWINGS

FIG. 1 is a graphic depiction of an embodiment of the invention having a flexible base and top memory foam section.

FIG. 2 is a graphic depiction of an embodiment of the invention used in sexual intercourse by a man and a woman.

FIG. 3 is a graphic depiction of an alternative embodiment of the invention in use with a man's penis.

FIG. 4 is a perspective view and graphic depiction of a man and a woman using the instant inventions during sexual intercourse.

FIG. 5 is an alternative perspective view and graphic depiction of a man and a woman using the instant inventions during sexual intercourse.

FIG. 6 is an additional alternative perspective view and graphic depiction of a man and a woman using the instant inventions during sexual intercourse.

FIG. 7 is a graphic depiction of an alternative embodiment of the invention having a first insertion section and a second continuous curved external lower section, the first section for insertion into the vagina, the second section for stimulating the clitoris.

FIG. 8 is a graphic depiction of an alternative embodiment of the invention as shown in FIG. 7, with a rechargeable battery and control unit.

FIG. 9 is a graphic depiction of an alternative embodiment of the invention as shown in FIG. 8, with a removable top section.

FIG. 10 is a graphic depiction of an alternative embodiment of the invention as shown in FIG. 8, with a vibrator replaced with a clitoral air flow unit.

FIG. 11 is a graphic depiction of an alternative embodiment of the invention as shown in FIG. 10, with a removable top section.

FIG. 12 is a graphic depiction of an alternative embodiment of the invention as shown in FIG. 10, with the clitoral air flow unit replaced with a clitoral pedal unit.

FIG. 13 is a graphic depiction of an alternative embodiment of the invention as shown in FIG. 12, with a removable top section.

FIG. 14 is a rear view graphic depiction of an embodiment of the invention.

FIG. 15 is a front view graphic depiction of the invention shown in FIG. 14.

FIG. 16 is a front view graphic depiction of the invention shown in FIG. 15, with a penis atop the device.

### DETAILED DESCRIPTION

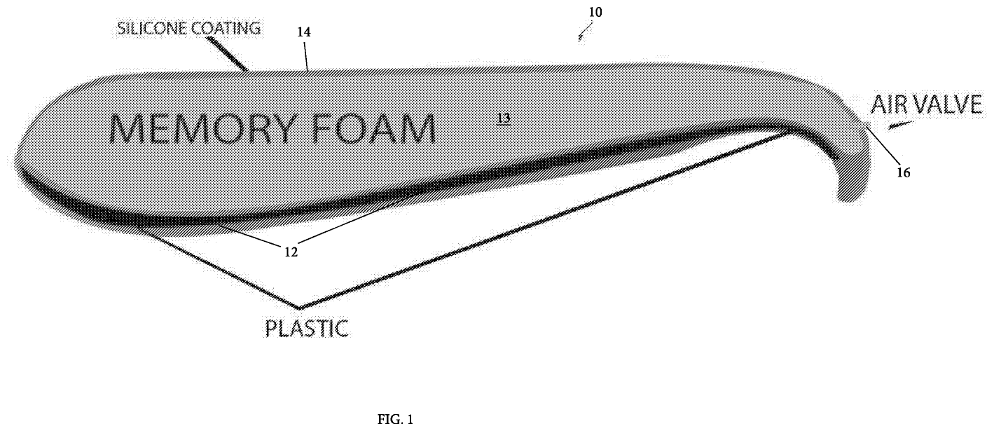

With reference to FIG. 1, an embodiment of the improved sexual aid device 10 is shown having a flexible base 12 and top memory foam section 13. The device includes a silicone coating 14 or equivalent to finish the product as suitable for use, and air valve 16 for use with an air bladder (not shown) which would replace the memory foam top section. Base 12 can be of plastic, composites or similar materials that are partially rigid and flexible, and is elongated in a generally spoon shaped to conform to a man's penis. Upper component 13 is attached to base 12, and this top section can be memory foam 13 or alternatively an air bladder as stated. This sexual aid may function as a solo sex stimulant or toy for one person, or for use with a couple during sex. The device can be inserted into the woman before the man enters, and thereafter the man will slide into the vagina on top of the foam or air bladder. Alternatively, the man can first place his penis onto the memory foam or air bladder before entering the vagina, and thereafter enter with both in place. This aid is used in conjunction with the male penis to create a tighter fit for the man, a greater feel for the woman, and enhanced sensation and pleasure for both.

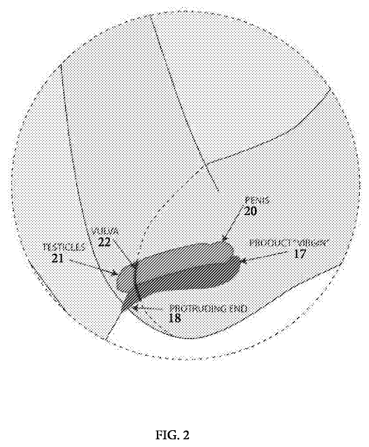

FIG. 2 is a graphic depiction of an embodiment of the invention as used in sexual intercourse by a man and a woman. The man's penis 20 is placed on the top section of the invention 17 shown in FIG. 1, the aid having protruding end 18. Both together are inserted into the vulva 22 and vagina. When inserted, testicles 21 and protruding end 18 remain external to the vagina.

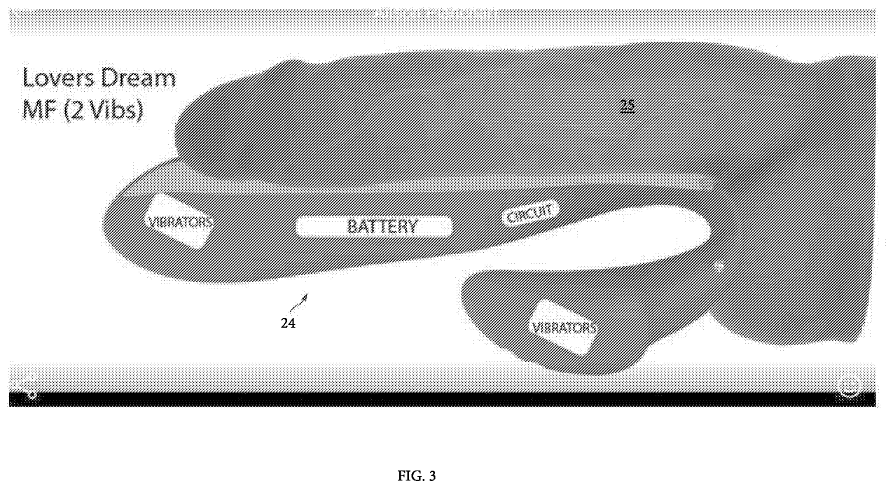

FIG. 3 is a graphic depiction of an alternative embodiment of sexual aid 24 in use with a man's penis 25. The penis lies on top of the aid, whether it is memory foam, an air bladder or removable top, as further described in detail hereinafter. This particular sexual aid is shown in FIG. 7 with a detailed description of the components.

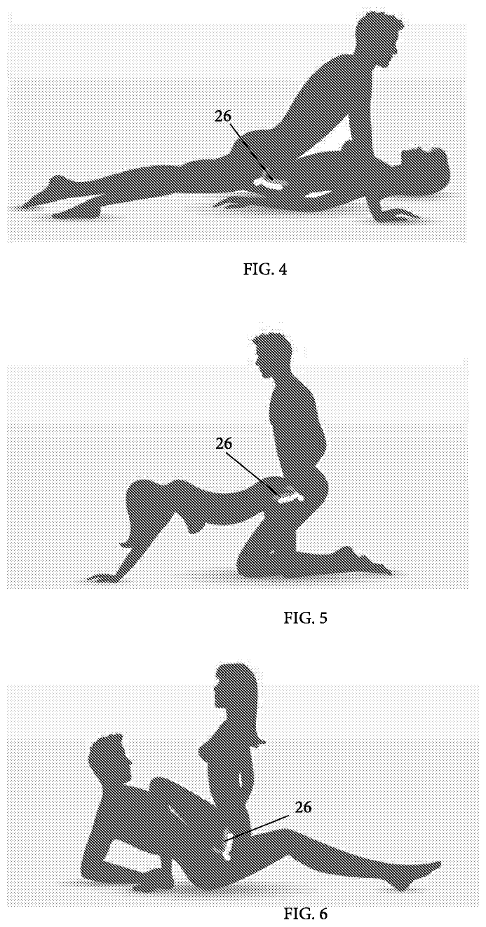

FIG. 4 is a perspective view and graphic depiction of a man and a woman using a sexual aid 26 of Applicant's invention during sexual intercourse in the missionary position.

FIG. 5 is an alternative perspective view and graphic depiction of a man and a woman using a sexual aid 26 of Applicant's invention during sexual intercourse in the male behind position.

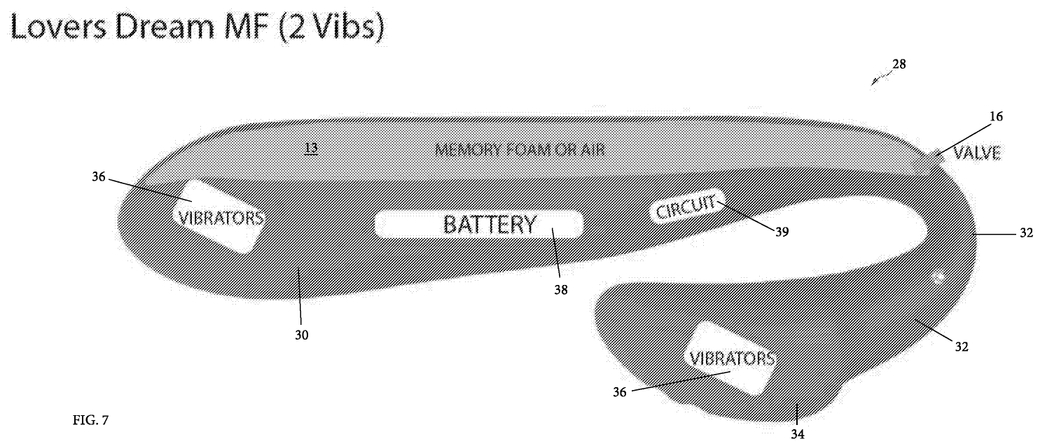

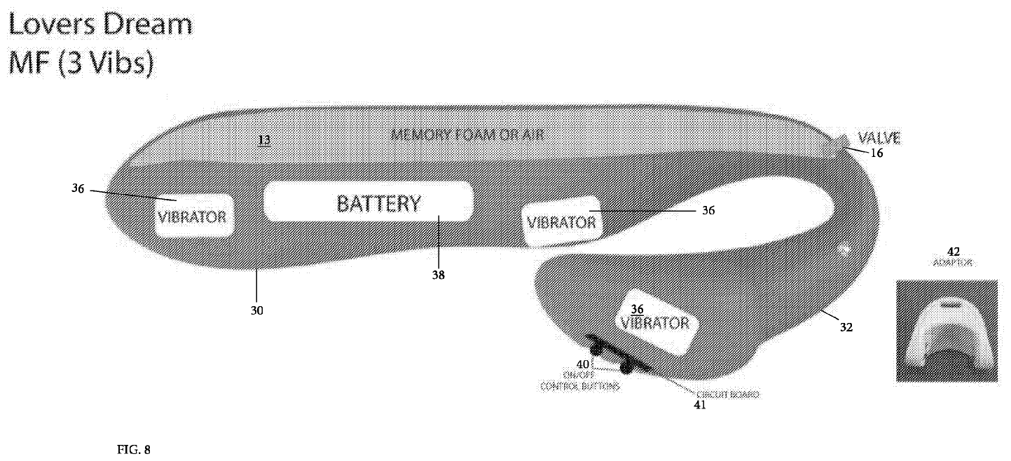

FIG. 6 is an additional alternative perspective view and graphic depiction of a man and a woman using a sexual aid 26 of Applicant's invention during sexual intercourse in the female-superior position.

FIG. 7 is a graphic depiction of an alternative embodiment of the sexual aid 28 having a first insertion section 30 and a second continuous curved external lower section 32, the first section 30 for insertion into the vagina, the second section 32 for stimulating the clitoris. The curved section 32 or finger member may have an enlarged end 34 and during sex, the finger member is designed to be positioned in front of the male testicles as shown in FIG. 3 and contacting the clitoris. When the sexual aid 28 is used in the sexual position shown in FIG. 5, the enlarged end 34 can alternatively be inserted into the woman's anus. Also shown is an air valve 16 for inflating a bladder or the use of memory foam 13 secured atop the base member. These devices also include one or more vibrators 36 selectively positioned about the aid, and control means for activation, and a removable or rechargeable battery 38 power source, and internal circuit 39 for enabling the vibrators.

FIG. 8 is a graphic depiction of the aid device as shown in FIG. 7, with a rechargeable battery 38 and control unit 40 that includes control buttons on/off functions and internal circuit board 41. Adapter 42 is used to recharge the battery with a dock fit connection. A remote control can be used with the sexual aid device, and communicate electronically with circuit 39, vibrators 36, and/or circuit board 41.

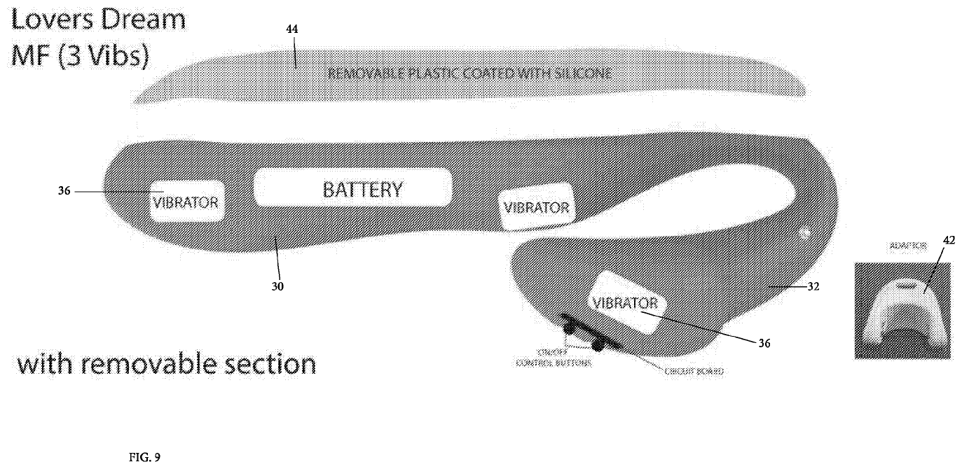

FIG. 9 illustrates the sexual aid shown in FIG. 8 with a removable top section 44 comprised of a plastic, composite, rubberized or similar material coated with silicone or other desired coating to finish the device for safe and comfortable use. This device can be used by a person in solo fashion, or by removing the top section with a man's penis thereon during sexual intercourse.

The improved sexual aid devices shown in FIGS. 7 through 13 having the vaginal insertion section and continuous external curved finger section all enhance the sexual experience for partners by simultaneously pleasuring both partners with penis, vaginal and clitoral stimulation by the various devices. This is enabled by the curvature arm of the devices and the external and internal sections, and the placement of vibration and stimulation components at proper locations as depicted in the Figures.

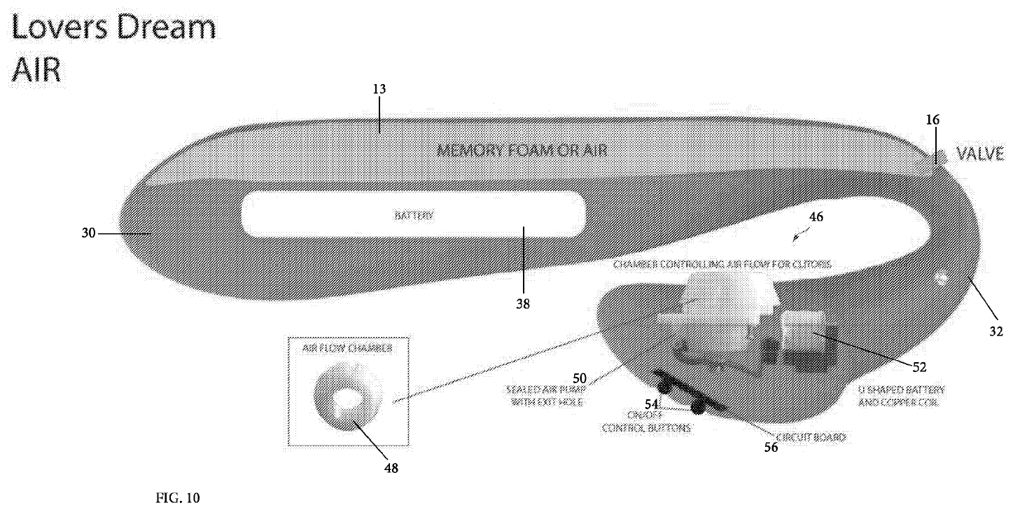

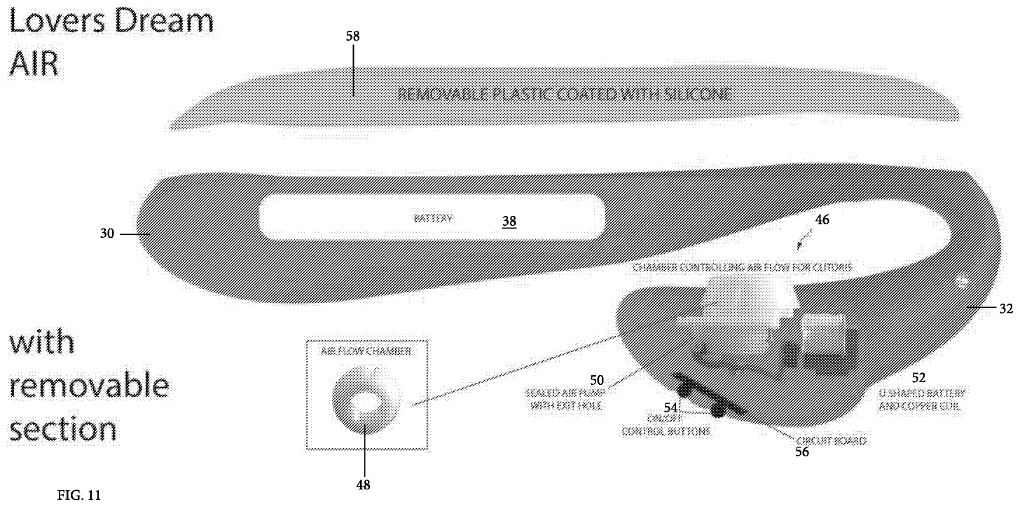

FIG. 10 is a graphic depiction of the sexual aid invention as shown in FIG. 8, with a vibrator 36 replaced with a clitoral air flow unit 46. This unit includes air flow chamber 48, sealed air pump 50, U shaped magnet and copper coil unit 52, as well as control on/off switches 54 and circuit board 56 for electronics. Air is pumped to the clitoris through the flow chamber 48. The pump can be driven by a magnet that changes polarity to move the piston arm as needed. When operated this clitoral unit has a diaphragm that pulsates forward and back, extending and retracting, creating waves of air flow onto and over the clitoral region, enhancing stimulation and pleasure. A sonic wave generator unit can also be used in the, replacing this unit, for similar effects in oscillations and pulsation operation.

FIG. 11 is a graphic depiction of an alternative embodiment of the sexual aid invention as shown in FIG. 10, with a removable top section 58, similar to those previously described.

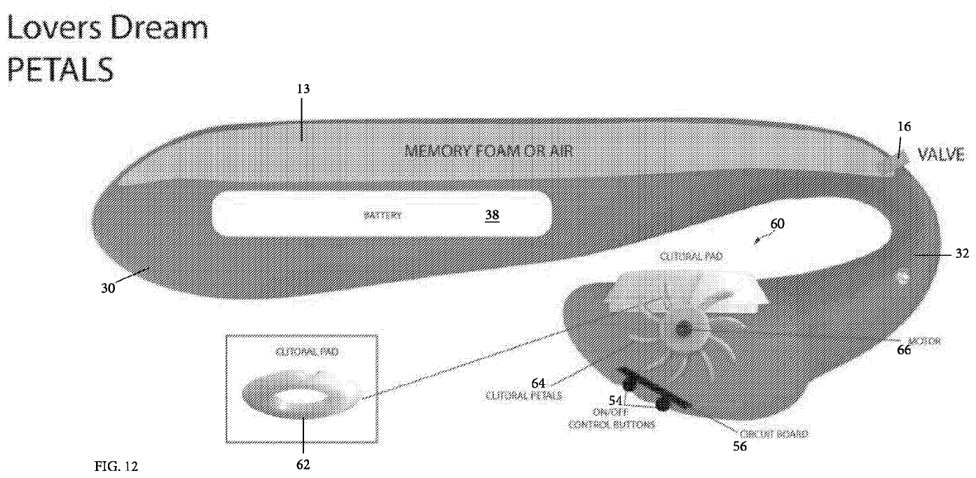

FIG. 12 is a graphic depiction of the sexual aid device as shown in FIG. 10, with the clitoral air flow unit 46 replaced with a clitoral pedal unit 60. Unit 60 includes clitoral pad 62, clitoral petals 64, drive mechanism 66, and the switches 54 and circuit board electronics 56. The clitoral pad is oval and positioned over the clitoris where the petals make contact. This device has pedals, rotating brushes, finger arms or the like which a motor within a housing rotates the assembly as a spinning mechanism. The pedals softly engage the clitoris in repeated sequential contacts, relatively high frequency fast motion, greatly stimulating the clitoris while the aid device simultaneously provides the vibration and other contact features of the insertion member, as previously described.

The battery is within the main body of the device, and where a charger is used, contacts can be pins through the body, pins inside an adapter, or a charging pad or dock can be utilized, as well as a remote control.

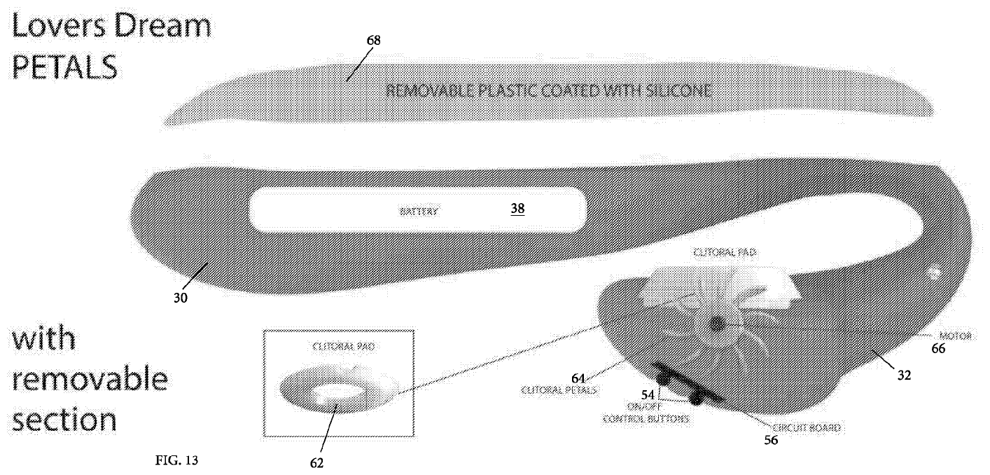

FIG. 13 is a graphic depiction the device as shown in FIG. 12, with a removable top section 68, as described above with other inventive devices.

In operation, the aid devices shown in FIGS. 10 through 13 can include one, two or three vibrators for the clitoris, the clitoris and G-spot, and/or the area deep within the vagina. There is also the option of the memory foam or air bladder for accommodating the male penis. The combined effect of the entire devices is realized fully by both partners in a unique, symbiotic and synergistic effect, in achieving greater, faster, stronger and prolonged orgasms.

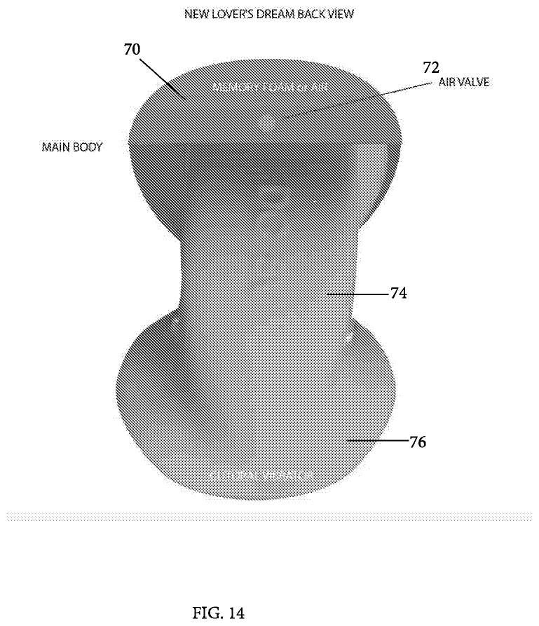

FIG. 14 is a rear view illustration of an embodiment of the invention depicting memory foam 70, air valve 72 when an air bladder is utilized, curved body arm/finger 74, and clitoral vibrator section 76.

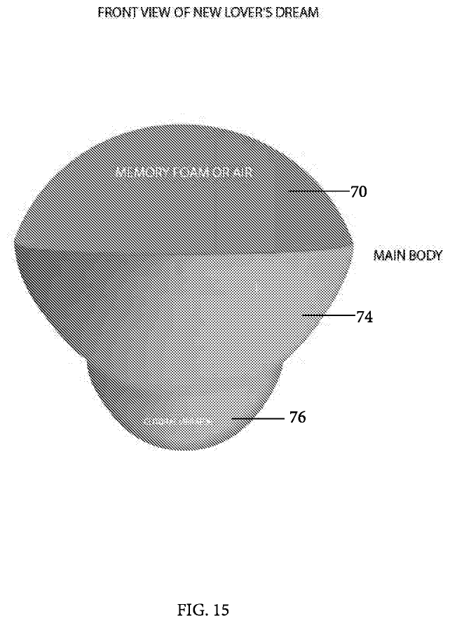

FIG. 15 is a front view illustration of the sex aid device shown in FIG. 14.

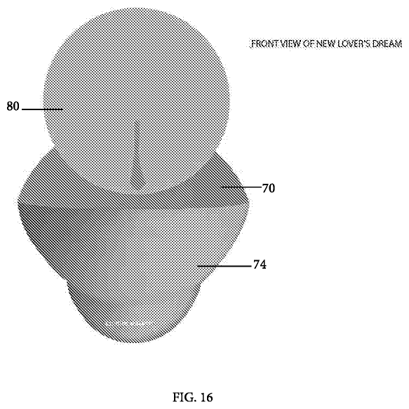

FIG. 16 is a front view graphic depiction of the invention shown in FIG. 15, with a penis 80 atop the device.

The instant invention has been shown and described herein in what is considered to be the most practical and preferred embodiments. It is recognized, however, that departures may be made therefrom within the scope of the invention and that obvious modifications will occur to a person skilled in the art.

What is claimed is:

1. An improved sexual aid device, comprising;
a semi-rigid base member, for insertion into a woman's vagina;
said semi-rigid flexible base member having a shape generally conforming to the shape of a male penis;
a top section secured to said semi-rigid base member;
said top section being a memory foam material; and
said top section for receiving said male penis.

2. An improved sexual aid device, comprising;
a semi-rigid base member, for insertion into a woman's vagina;
said semi-rigid flexible base member having a shape generally conforming to the shape of a male penis;
a top section secured to said semi-rigid base member;
said top section being an inflatable bladder;
said inflatable bladder have an input valve; and
said inflatable bladder for receiving said male penis.

3. An improved sexual aid device, comprising;
a first insertion member;
a second curved external member, said second curved external member being a continuous extension from said first insertion member;
said first insertion member for insertion into a woman's vagina;
said second curved external member for stimulating a woman's clitoris;
said second curved external member including a stimulation means; and
said stimulation means including electronic control means.

4. The apparatus of claim 3, wherein said first insertion member includes a top section having a memory foam; and
said top section for receiving a male penis.

5. The apparatus of claim 3, wherein said first insertion member includes a top section having an inflatable bladder; and
said top section for receiving a male penis.

6. The apparatus of claim 3, wherein said first insertion member includes a top section having a removable member; and
said top section for receiving a male penis.
